(19) United States
(12) Patent Application Publication
(10) Pub. No.: US 2023/0310468 A1
(43) Pub. Date: Oct. 5, 2023

(54) Parenteral pharmaceutical composition comprising cyclophosphamide and a mixture of liquids

(71) Applicant: Sandoz AG, Basel (CH)

(72) Inventors: Katri Rouvali, Kundl (AT); Réka Kangyal, Kundl (AT); Katja Foerster, Kundl (AT); Stephan Hold, Kundl (AT); Reinhard Pell, Kundl (AT); Peter Finichiu, Kundl (AT); Florian Zauner, Kundl (AT); Florian Huber, Kundl (AT)

(73) Assignee: Sandoz AG, Basel (CH)

(21) Appl. No.: 18/021,100

(22) PCT Filed: Aug. 16, 2021
(86) PCT No.: PCT/EP2021/072674

§ 371 Date: Feb. 13, 2023

(30) Foreign Application Priority Data
Aug. 17, 2020 (EP) 20191374.6

(51) Int. Cl. A61K 31/675; A61K 47/26; A61K 9/00; A61K 9/08

(52) U.S. Cl. CPC A61K 31/675; A61K 47/26; A61K 9/0019; A61K 9/08

(57) ABSTRACT

The present invention is directed to a parenteral pharmaceutical composition comprising cyclophosphamide and a mixture of specific liquids in specific amounts, respectively.

### FIELD OF THE INVENTION

The present invention is directed to a parenteral pharmaceutical composition comprising cyclophosphamide and a mixture of specific liquids in specific amounts, respectively.

### BACKGROUND OF THE INVENTION

Cyclophosphamide (IUPAC name N,N-bis(2-chloroethyl)-2-oxo-1,3,2λ⁵-oxazaphosphinan-2-amine and also referred to as cytophosphane) is a synthetic active pharmaceutical agent and belongs to the compounds with an alkylating effect. Cyclophosphamide (tradename Endoxan) was developed in the 1950s and is used inter alia for the treatment of various cancerous malignances and as an immune suppressor for example in the treatment of nephrotic syndrome, granulomatosis with polyangiitis and following organ transplant.

Cyclophosphamide can be taken by mouth, but it is commonly administered by intravenous (i.v.) infusion, intravenous injection or subcutaneous (s.c.) injection due to low oral bioavailability as well as the instability of the compound.

After being absorbed, cyclophosphamide (which can be considered as a prodrug) is activated in the liver and after that the resulting compound (4-hydroxycyclo-phophonamide) becomes cytotoxic. Thus, the main effect of cyclophosphamide is due to its metabolite phosphoramide mustard. This metabolite is just formed in cells that have low levels of ALDH (aldehyddehydrogenase). Phosphoramide mustard forms DNA crosslinks both between and within DNA strands at guanine N-7 positions. This is irreversible and leads to cell apoptosis.

Cyclophosphamide can induce beneficial immunomodulatory effects in adaptive immunotherapy, wherein the suggested mechanisms include a) elimination of T regulatory cells in naive and tumor-bearing hosts, b) induction of T cell growth factors, such as type I IFNs, and/or c) enhanced grafting of adoptively transferred, tumor-reactive effector T cells by the creation of an immunologic space niche.

Cyclophosphamide is reported to be prone to degradation in several solvents at room temperature and/or to at least partial precipitation from the solvent.

US 2015/0320775 A1 describes a cyclophosphamide-containing composition comprising cyclophosphamide, ethanol and an ethanol-soluble acidifying agent, in particular citric acid.

Nevertheless, cyclophosphamide usually is provided as a lyophilizate or sterile powder in a vial that needs a reconstitution step before the solution is ready to use, i.e. in the prior art there is the need to prepare the formulation in situ before treatment. This requires vigorous shaking to prepare a solution for i.v. administration or a uniform suspension for s.c. administration and visual inspection of the formulation prior to administration. If the suspension or solution is not uniform it must be discarded, and the process must be repeated. Accordingly, the reconstitution procedure is labor and time intensive and can further result in unnecessary dosing errors as well as the risk of potential contamination. In addition, it is only possible to administer the cyclophosphamide at low concentrations. Thus, another possible drawback of the cyclophosphamide formulations of the prior art is the high incidence of injection site reactions, in particular if administered s.c.

Additionally, the reconstitution step for forming a suspension can lead to agglomeration of particles resulting in a dosing that is difficult and unpredictable as the particles are distributed unevenly in the suspension. Due to possible re-agglomeration of the particles after the reconstitution step, the presently available suspension should not be stored for more than 1 hour at 25° C. after reconstitution. This results in a logistically cumbersome administration process, as the reconstituted formulation must be brought and administered to the patient within 1 hour after reconstitution at a compounding station. Therefore, it was an object of the present invention to provide a pharmaceutical cyclophosphamide-containing formulation that is a ready-to-use formulation while maintaining high stability at room temperature. In addition, it was another object of the present invention to provide a pharmaceutical cyclophosphamide-containing formulation that is highly concentrated, therefore minimizing the chances of adverse reactions at the injection site. Moreover, a cyclophosphamide-containing formulation avoiding hazardous personnel exposure, long preparation time and potential mistakes during reconstitution should be provided.

### SUMMARY OF THE INVENTION

At least one of these objects has surprisingly been solved by the parenteral pharmaceutical composition comprising (a) cyclophosphamide and (b) a mixture of liquids, wherein the mixture of liquids (b) comprises (b1) 60 to 99.5 vol % ethanol, (b2) 0 to 25 vol % of an organic solvent selected from glycerol, ethylene glycol, polyethylene glycol, propylene glycol and mixtures thereof and (b3) 0 to 20 vol % of liquid surfactant.

It has surprisingly been found that a stable, highly concentrated composition can be provided by dissolving cyclophosphamide in the above-indicated liquid mixture. This finding was in particular unexpected as a ternary mixture of solvents is rarely used in pharmaceutical compositions due to the hardly predictable behavior of mixtures with several components.

In another aspect, the invention relates to an injection syringe, a container or a vial containing the present pharmaceutical composition for single or multiple dosing.

In a further aspect, the present invention relates to a kit of parts comprising the present pharmaceutical composition in a vial, cartridge or container and an injection device.

In yet another aspect, the present invention relates to the pharmaceutical composition of the present invention for use in the treatment of cancer as well as nephrotic syndrome, granulomatosis with polyangiitis and following organ transplant.

Finally, the present invention relates to a method for preparing the present parenteral pharmaceutical composition comprising the steps of (i) mixing liquids (b1), (b2) and (b3) and (ii) adding cyclophosphamide (a) to the mixture from step (i) to obtain the parenteral pharmaceutical composition.

### DETAILED DESCRIPTION

The following definitions are relevant in connection with the embodiments of the present invention.

The term “parenteral” preferably refers to a composition that is not orally administered. In particular, a parenteral composition is suitable for injection or infusion, especially for administration by intravenous injection, intravenous infusion, intramuscular injection and/or subcutaneous injection.

The term “pharmaceutical” preferably refers to compositions, wherein the active ingredient and liquids (b1) to (b3) fulfil all necessary pharmaceutical standards/monographs of the USP-NF (United States Pharmacopeia National Formulary) and/or Ph. Eur. (European Pharmacopoeia) with regard to purity of the active ingredient and/or amount of pathogens and/or bacterial contamination.

Accordingly, the ethanol used in the present invention preferably fulfills the requirements of Ph. Eur. monographs 4.00/1317 or 4.00/1318. Further, glycerol, if used in the present invention, preferably fulfills the requirements of Ph. Eur. monographs 4.00/0497 or 4.00/0496 and propylene glycol, if used in the present invention, preferably fulfills the requirements of Ph. Eur. monographs 4.00/0430. Further, the term “pharmaceutical composition” encompasses a composition fulfilling Ph. Eur. standards/monographs 4.00/2.06.08.00 and 4.002.06.14.00, which relate to the measurement and amounts of pyrogens and bacterial endotoxins, respectively.

The term “cyclophosphamide” preferably refers to cyclophosphamide which is a mixture of the (R)-form and the (S)-form of the molecule as represented by following Formula.

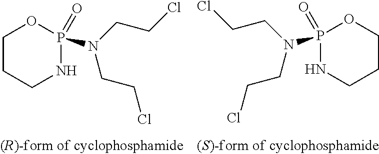

However, “cyclophosphamide” may also refer to enantiomers, polymorphs, hydrates, solvates and mixtures thereof. The term “hydrate” refers to cyclophosphamide to which water is complexed by intermolecular forces in the crystal lattice of the cyclophosphamide (before dissolution in the mixture of liquids (b1) to (b3)). The term “solvate” refers to cyclophosphamide complexed with a solvent molecule other than water that is bound by intermolecular forces in the crystal lattice of the cyclophosphamide (before dissolution in the mixture of liquids (b1) to (b3)).

The meaning of the term “comprising” is to be interpreted as encompassing all the specifically mentioned features as well optional, additional, unspecified ones, whereas the term “consisting of” only includes those features as specified. Therefore, “comprising” includes as a limiting case the composition specified by “consisting of”.

The term “vol.-%” refers to the amount of the respective concentration of a certain solute (in the present case “liquid”), measured by volume, in a solution (in the present case “mixture of liquids”).

Preferred embodiments according to the invention are defined hereinafter. The preferred embodiments are preferred alone or in combination. Further, it is to be understood that the following preferred embodiments refer to all aspects of the present invention, i.e. the pharmaceutical composition, the injection syringe, cartridge, container or vial containing the composition, the composition for use, the kit of parts and the method for preparing the pharmaceutical composition.

In an embodiment, the pharmaceutical composition of the present invention consists of cyclophosphamide and the mixtures of liquids comprising liquids (b1) to (b3) in the respective amounts. Thus, it is preferred that the pharmaceutical composition does not comprise any excipients, buffers, additional antimicrobial agents, surfactants or solubility enhancers.

In an embodiment, the pharmaceutical composition is essentially free of water. The term “essentially free” of water refers to a pharmaceutical composition that contains less than 0.8 wt.-% water, preferably less than 0.5 wt.-% water or more preferably less than 0.12 wt.-% water, or particularly preferred less than 0.10 wt.-% water. As the water content may potentially vary during storage, it is preferred that the pharmaceutical composition contains less than the afore-mentioned water content at any point during the storage period. The water content can be measured by Karl Fischer titration with e.g. a Mettler DL 18 titrator.

In another embodiment, the pharmaceutical composition is a solution. Herein, a solution can be regarded as a special type of homogeneous mixture composed of two or more substances. In such a mixture, a solute (in the present case “cyclophosphamide”) is the substance dissolved in another substance (in the present case “mixture of liquids (b1) to (b3)”), known as a solvent. Whether the solute is completely dissolved in the solvent can be determined by visual inspection. A solution allows for the administration by intravenous infusion or injection as well as subcutaneous injection. However, it is also possible to provide the pharmaceutical composition as a suspension. Said suspension preferably might be used for subcutaneous injection.

In an embodiment, the composition is sterile in accordance with Ph. Eur. 4.00/2.06.01.00 and/or 4.00/5.01.01.00.

In an embodiment, the composition is sterile, i.e. it contains less than 10⁻⁴ wt.-% of nonsterile material, such as microbial contamination. Sterility of the composition can be ensured by a number of processes, such as thermal, chemical, radiation and filtration processes. In the present invention, it is preferred that a nonthermal process is employed. Thus, it is preferred that sterilization is ensured by either gamma ray or UV light irradiation or by filtration through a suitable membrane filter. Suitable filters are known to the skilled person and are described in “The Theory and Practice of Industrial Pharmacy”, Lachmann et al., 3^{rd} edition, Varghese Publishing house. Examples include cellulose filters, Nylon 66, polytetrafluoroethylene with polyethylene or polypropylene substrate. The membranes of the filters usually have a nominal pore size of 0.22 μm or less, preferably 0.20 μm or less, more preferably 0.10 μm or less.

In a preferred embodiment, the pharmaceutical composition has a storage stability at 5° C. of up to 200 days, up to 300 day, up to 350 days, up to 400 day or up to 500 days, up to 600 day or up to 750 days.

In an embodiment, the pharmaceutical composition has a storage stability at 5° C. of up to 6 months, up to 12 months, up to 15 months, up to 18 months or up to 24 months.

The term “storage stability” refers to a composition, wherein cyclophosphamide does not change, deteriorate, react or decompose over the prescribed time period by more than 5 wt.-%, more than 4 wt.-%, more than 3 wt.-%, more than 2 wt.-%, more than 1.5 wt.-%, more than 1 wt.-%, more than 0.5 wt.-% or more than 0.1 wt.-% based on the total amount of the respective component. It is preferred that the term storage stability refers to a composition wherein the cyclophosphamide does not change, deteriorate, react or decompose over the prescribed time period by more than 5 wt.-%.

In an embodiment, the present pharmaceutical composition comprises cyclophosphamide at a concentration of from 50 mg/mL to 350 mg/mL, of from 60 mg/mL to 330 mg/mL, of from 70 mg/mL to 310 mg/mL, of from 80 mg/mL to 300 mg/mL, of from 90 mg/mL to 290 mg/mL, of from 100 mg/mL to 280 mg/mL, of from 110 mg/mL to 270 mg/mL, of from 120 mg/mL to 260 mg/mL, of from 130 mg/mL to 260 mg/mL, of from 140 mg/mL to 250 mg/mL, of from 150 mg/mL to 240 mg/mL, of from 160 mg/mL to 230 mg/mL, of from 170 mg/mL to 220 mg/mL, of from 180 mg/mL to 210 mg/mL, or from 190 mg/mL to 200 mg/mL, preferably at a concentration of from 100 mg/mL to 250 mg/mL.

In an even more preferred embodiment, the pharmaceutical composition comprises cyclophosphamide at a concentration of 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL or 200 mg/mL, in particular of about 110 mg/mL.

In a particularly preferred embodiment, the present invention relates to a parenteral pharmaceutical composition comprising cyclophosphamide and mixture of liquids (b), wherein the pharmaceutical composition comprises cyclophosphamide at a concentration of 75 mg/mL to 125 mg/mL.

In another particularly preferred embodiment, the present invention relates to a parenteral pharmaceutical composition comprising cyclophosphamide and mixture of liquids (b), wherein the pharmaceutical composition comprises cyclophosphamide at a concentration of 100 mg/mL to 250 mg/mL.

In a particularly preferred embodiment, the present invention relates to a parenteral pharmaceutical composition consisting of cyclophosphamide and mixture of liquids (b), wherein the pharmaceutical composition comprises cyclophosphamide at a concentration of 150 mg/mL to 200 mg/mL.

In preferred embodiment the mixture of liquids (b) comprises
(b1) 60 to 99.5 vol.-%, preferably 70 to 95 vol.-%, in particular 80 to 90 vol.-% of ethanol,
(b2) 0 to 25 vol.-%, preferably 1 to 22 vol.-%, more preferably 2 to 9 vol.-%, in particular about 8 vol.-% of an organic solvent selected from glycerol, ethylene glycol, polyethylene glycol, propylene glycol and mixtures thereof, and
(b3) 0 to 20 vol.-%, preferably 1 to 15 vol.-%, more preferably 2 to 9 vol.-%, in particular about 8 vol.-% of liquid surfactant.

In an alternatively preferred embodiment the mixture of liquids (b) comprises just two liquids, preferably (b1) ethanol and (b2) an organic solvent selected from glycerol, ethylene glycol, polyethylene glycol, propylene glycol and mixtures thereof.

In an embodiment the mixture of liquids (b) comprises
(b1) 75 to 99.5 vol.-%, preferably 78 to 90 vol.-%, in particular about 80 vol.-% of ethanol,
(b2) 0.5 to 25 vol.-%, preferably 10 to 22 vol.-%, in particular about 20 vol.-% of an organic solvent selected from glycerol, ethylene glycol, polyethylene glycol, propylene glycol and mixtures thereof.

The mixture of liquids (b) comprises an organic solvent (b2) selected from glycerol, ethylene glycol, propylene glycol and mixtures thereof, preferably selected from glycerol, propylene glycol and mixtures thereof. Particularly preferred as organic solvent (b2) is propylene glycol.

The mixture of liquids (b) comprises a liquid surfactant (b3). Within this application a liquid surfactant is referred to as a surfactant which is in liquid state at 23° C. and at 101325 Pa (normal pressure). Surfactants are well known in the art and they for example can be used to prevent aggregation of molecules in a formulation. In a preferred embodiment the liquid surfactant (b3) is a non-ionic surfactant. Non-limiting examples of surfactants are polysorbates (Tween®) such as polyoxyethylen-(20)-sorbitanmonolaurate (Polysorbat 20) polyoxyethylen-(4)-sorbitanmonolaurate (Polysorbat 21), polyoxyethylen-(20)-sorbitanmonopalmitate (Polysorbat 40), polyoxyethylen-(20)-sorbitanmonostearate (Polysorbat 60), polyoxyethylen-(4)-sorbitanmonostearaet (Polysorbat 61), polyoxyethylen-(20)-sorbitantristearate (Polysorbat 65), polyoxyethylen-(20)-sorbitanmonooleate (Polysorbat 80), polyoxyethylen-(5)-sorbitanmonooleate (Polysorbat 81), polyoxyethylen-(20)-sorbitantrioleate (Polysorbat 85) and polyoxyethylen-(20)-sorbitanmonoisostearate (Polysorbat 120); and polyalkylene glycol ether such as polyoxyethylene (2) lauryl ether, polyehtylene (10) monodecylether, polyoxy ethylene (10) tridecylether, preferably polyoxyethylen-(20)-sorbitanmonopalmitate (Polysorbat 40), polyoxyethylen-(20)-sorbitanmonostearate (Polysorbat 60), and polyoxyethylen-(20)-sorbitanmonooleate (Polysorbat 80). Particular preferred as liquid surfactant is poly-oxyethylen-(20)-sorbitanmonooleate (Polysorbat 80).

In a preferred embodiment the parenteral pharmaceutical composition comprises (a) cyclophosphamide and (b1) 60 to 99.5 vol.-% of ethanol, (b2) 0 to 25 vol.-% of propylene glycol and (b3) 0 to 20 vol.-% of polyoxyethylen-(20)-sorbitanmonooleate (Polysorbat 80).

In a preferred embodiment the parenteral pharmaceutical composition comprises (a) cyclophosphamide and (b1) 60 to 99.5 vol.-% of ethanol, (b2) 0 to 25 vol.-% of propylene glycol and (b3) 0 to 20 vol.-% of polyoxyethylen-(20)-sorbitanmonooleate (Polysorbat 80), wherein the parenteral pharmaceutical composition comprises (a) cyclophosphamide in a concentration of 50 mg/mL to 350 mg/mL.

In a preferred embodiment the parenteral pharmaceutical composition comprises (a) cyclophosphamide and (b1) about 80 vol.-% of ethanol and (b2) about 20 vol.-% of propylene glycol, wherein the parenteral pharmaceutical composition comprises (a) cyclophosphamide in a concentration of 50 mg/mL to 150 mg/mL, in particular about 100 mg/mL.

In an embodiment, the parenteral pharmaceutical composition is a ready-to-use composition for injection or infusion or a concentrate for infusion, preferably a concentrate for infusion. The concentrate may be useful for administration by infusion. Thus, the pharmaceutical composition of the present invention does not need to be manually prepared in situ and can be used as is without an additional reconstitution step.

The present pharmaceutical composition may be used to treat animals. Thus, in an embodiment the present pharmaceutical composition is a veterinarian composition. The animals to be treated by the present pharmaceutical composition may be mammals. Non-limiting examples of mammals include livestock such as cattle, pigs and horses as well as pets such as cats and dogs.

In another aspect, the invention relates to an injection syringe, a cartridge, a container or a vial containing the pharmaceutical composition of the present invention for single or multiple dosing. The container or vial can be made of glass or plastic, preferably glass. Examples for suitable plastic materials comprise polyethylene, polypropylene, polyvinyl chloride, polycarbonate, polyamide polystyrene and polytetrafluoroethylene. These plastic materials may contain further additives, such as colorants, stabilizers and antioxidants. Examples for glass are soda-lime glass and borosilicate. The composition may be removed from the container or vial by either breaking the vial or container at a predetermined breaking point or by providing the vial or container with a closure. Preferably the container or vial is provided with a closure. Said closure may be a septum or a rubber stopper and a crimp cap, wherein the septum or a rubber stopper can be punctured by a needle. Examples of suitable rubber septa/stoppers are butyl rubbers, neoprene rubber, silicone rubbers. In order to prevent moisture absorption, the rubber stopper may be coated by a hydrophobic coating agent, such as silicone polymers or may contain a desiccant. It is particularly preferred that the container or vial is filled using a blow-fill-seal (BFS) technology wherein the container or vial is molded, aseptically filled and sealed in a continuous process.

The use of pre-filled syringes or cartridges for syringe systems reduces the degree of manipulation required and therefore further facilitates easy administration.

In a particularly preferred embodiment, each injection syringe, cartridge, container or vial has a uniform content of cyclophosphamide in accordance with Ph. Eur. 4.00/2.09.06.00.

In a preferred embodiment, the injection syringe, cartridge, container or vial contains a nominal volume of 0.2 mL, 0.3 mL, 0.4 mL, 0.5 mL, 1.0 mL, 1.5 mL, 2.0 mL, 5.0 mL, 7.5 mL, 10 mL, 12.5 mL, 15 mL, 15 mL, 17.5 mL, 20 mL, 25 mL of the pharmaceutical composition. It is even more preferred that the injection syringe, cartridge, container or vial is a single-dose injection syringe, cartridge, container or vial. The term “nominal” volume refers to the volume as intended but the actual volume may deviate by an amount of ±5 volume % from the nominal volume. In a preferred embodiment, the term “nominal” volume in each vial complies with Ph. Eur. 4.00/2.09.17.00 regarding the amount of extractable volume.

In a preferred embodiment, the pharmaceutical composition is contained in a vial. It is even more preferred that the pharmaceutical composition is contained in a single-dose or a multi-dose vial. A single-dose vial can contain sufficient composition for administrations over several hours. It is preferred that the single-dose vial contains the pharmaceutical composition in an amount sufficient for at least one day. The single-dose vial may contain the pharmaceutical composition in an amount of from 1.0 mL to 50 mL, preferably in an amount of from 5.0 to 20 mL. A multi-dose vial can contain sufficient composition for administrations over several days. It is preferred that the multi-dose vial contains the pharmaceutical composition in an amount sufficient for at least seven days. The multi-dose vial may contain the pharmaceutical composition in an amount of from 10.0 mL to 250 mL, preferably in an amount of from 35.0 to 100 mL. Preferred is a single-dose vial.

In a preferred embodiment, the parenteral pharmaceutical composition comprises (a) cyclophosphamide, (b1) 60 to 99.5 vol % ethanol, (b2) 0 to 25 vol % of propylene glycol and (b3) 0 to 20 vol % polyoxyethylene (20)-sorbitan monolaurate in a multi-dose vial, wherein the pharmaceutical composition comprises cyclophosphamide at a concentration of 100 mg/mL to 250 mg/mL and wherein the multi-dose vial contains 1.0 to 50 mL of the pharmaceutical composition.

In a preferred embodiment, the parenteral pharmaceutical composition comprises (a) cyclophosphamide, (b1) about 80 vol % ethanol, (b2) about 20 vol % of propylene glycol in a multi-dose vial wherein the pharmaceutical composition comprises cyclophosphamide at a concentration of 50 mg/mL to 150 mg/mL, in particular about 100 mg/mL, and wherein the multi-dose vial contains 1.0 to 50 mL of the pharmaceutical composition.

Further, the invention relates to a kit of parts comprising the pharmaceutical composition in a vial, cartridge or container and an injection device. It is preferred that the kit of parts comprises the present parenteral pharmaceutical composition in a vial and an injection device such as a syringe.

Further, the invention relates to the present parenteral pharmaceutical composition for use in the treatment of cancer as well as nephrotic syndrome, granulomatosis with polyangiitis and following organ transplant.

Preferably the cancer may be selected from lymphoma, multiple myeloma, leukemia, ovarian cancer, breast cancer, small cell lung cancer and sarcoma.

In an embodiment, the pharmaceutical composition is administered systemically. The term systemically refers to the delivery in the circulatory system of the subject and thus inherently affects its whole body. Examples of systemic administration include intramuscular (i.m.), intravenous (i.v.), intradermal (i.d.), transdermal (t.d.) and subcutaneous (s.c.). In an embodiment, the pharmaceutical composition is administered by subcutaneous (s.c.) or by intravenous (i.v.) injection or infusion.

In a particularly preferred embodiment, the present invention relates to a parenteral pharmaceutical composition comprising (a) cyclophosphamide and (b) a mixture of liquids comprising liquids (b1) to (b3) for use in the treatment of cancer as well as nephrotic syndrome, granulomatosis with polyangiitis and following organ transplant, preferably for use in the treatment of cancer, wherein the pharmaceutical composition comprises cyclophosphamide at a concentration of 100 mg/mL to 250 mg/mL and wherein the pharmaceutical composition is administered by intravenous (i.v.) injection or infusion.

In a particularly preferred embodiment, the present invention relates to a parenteral pharmaceutical composition consisting of (a) cyclophosphamide and (b) a mixture of liquids consisting of liquids (b1) to (b3) for use in the treatment of cancer as well as nephrotic syndrome, granulomatosis with polyangiitis and following organ transplant, preferably for use in the treatment of cancer, wherein the pharmaceutical composition comprises cyclophosphamide at a concentration of 100 mg/mL to 250 mg/mL and wherein the pharmaceutical composition is administered by intravenous (i.v.) injection or infusion.

In a particularly preferred embodiment, the present invention relates to a parenteral pharmaceutical composition comprising (a) cyclophosphamide and (b) a mixture of liquids comprising (b1) ethanol and (b2) propylene glycol for use in the treatment of cancer as well as nephrotic syndrome, granulomatosis with polyangiitis and following organ transplant, preferably for use in the treatment of cancer, wherein the pharmaceutical composition comprises cyclophosphamide at a concentration of 75 mg/mL to 125 mg/mL and wherein the pharmaceutical composition is administered by intravenous (i.v.) injection or infusion.

In a particularly preferred embodiment, the present invention relates to a parenteral pharmaceutical composition consisting of (a) cyclophosphamide and (b1) ethanol and (b2) propylene glycol for use in the treatment of cancer as well as nephrotic syndrome, granulomatosis with polyangiitis and following organ transplant, preferably for use in the treatment of cancer, wherein the pharmaceutical composition comprises cyclophosphamide at a concentration of 75 mg/mL to 125 mg/mL and wherein the pharmaceutical composition is administered by intravenous (i.v.) injection or infusion.

In case the present pharmaceutical composition is administered by intravenous (i.v.) infusion, the present pharmaceutical composition is preferably mixed with an infusion medium. Suitable infusion media are for example electrolyte solutions selected from the group consisting of 0.9 wt.-% aqueous NaCl solution, 5 wt.-% aqueous glucose solution, Ringer's solution, Ringer's lactate solution and Ringer's acetate solution. Thus, it is preferred that the pharmaceutical composition is injected into an infusion medium to form a mixture of infusion medium and pharmaceutical composition which is administered to a patient. Administration of this mixture can be performed through a peripheral venous catheter. Preferably, the infusion medium has a volume of 50 to 100 mL.

In an aspect, the invention relates to a method for preparing the present pharmaceutical composition, comprising the steps of
(i) preparing a mixture of liquids (b) and
(ii) adding cyclophosphamide (a) to the mixture of liquids (b) obtained in step (i) to provide the parenteral pharmaceutical composition.

In step (i) the mixture of liquids (b) is prepared. As far as the components comprised in the mixture and the amounts/concentrations of these components are concerned, the same as described above applies.

Preferably, step (i) comprises mixing the components and homogenizing the resulting mixture of liquids. Homogenizing can comprise mechanical treatment of the mixture such as stirring and/or ultrasonic treatment, preferably stirring.

In step (ii) cyclophosphamide (a) is added to the mixture of liquids (b) obtained in step (i). Preferably, the resulting mixture is homogenized, wherein homogenizing is defined as described above, until dissolution, preferably complete dissolution, of cyclophosphamide. Complete dissolution of cyclophosphamide results in a solution which can be confirmed by visual inspection.

In a preferred embodiment step (i) and/or step (ii) can be carried out at a temperature of between 10° C. and 35° C., preferably of between 15° C. and 35° C., in particular at about 23° C. (room temperature).

In an embodiment, the present method further comprises the step of
(iii) sterilization of the pharmaceutical composition.

Sterilization of the pharmaceutical composition can be carried out by any means for sterilization known in the art. Preferably sterilization is performed via by gamma ray irradiation and/or by filtration through a suitable membrane filter. It is preferred that sterilization takes place by filtration such as filtration through a sterile membrane filter with a suitable pore size.

It is further preferred that the method further comprises the step of
(iv) filling the pharmaceutical composition as obtained from step (ii) or from step (iii), preferably as obtained from step (iii), into a syringe, cartridge, vial or container, preferably a container or vial, and sealing the syringe, cartridge, vial or container.

The syringe, cartridge, vial or container have preferably been sterilized before the filling in of the composition and the subsequent sealing. Sterilization can be carried by any means known in the art such by saturated steam, superheated steam, hydrogen peroxide and/or heat treatment, preferably by heat treatment.

Sealing a syringe, cartridge, vial or container can by carried out by any means known in the art. It is preferred that the composition as obtained from step (ii) or from step (iii) is filled in a vial or container, preferably a vial, sealed with a rubber stopper and a crimp cap.

It is preferred that all steps of the present method are performed under sterile conditions to provide a sterile pharmaceutical composition in accordance with Ph. Eur. 4.00/2.06.01.00 and/or 4.00/5.01.01.00.

In a preferred embodiment, the method for preparing the present pharmaceutical composition comprises the steps of
(i) preparing a mixture of liquids (b) and
(ii) adding cyclophosphamide (a) to the mixture of liquids (b) obtained in step (i) to provide the parenteral pharmaceutical composition,
(iii) sterilization of the pharmaceutical composition,
(iv) filling the pharmaceutical composition as obtained from step (ii) or from step (iii), into a syringe, cartridge, vial or container, preferably a container or vial, and sealing the syringe, cartridge, vial or container,
   wherein all steps are performed under sterile conditions to provide a sterile pharmaceutical composition in accordance with Ph. Eur. 4.00/2.06.01.00 and/or 4.00/5.01.01.00.

The manufacture of sterile pharmaceutical compositions is inter alia described in “Rule and Guidance for Pharmaceutical Manufacturers and Distributors” (The Orange Guide) by the Medicines and Healthcare products Regulatory Agency, published by Pharmaceutical Press, 2017.

### EXAMPLES

### Example 1: Stability Testing

Cyclophosphamide-containing solutions (samples A to G and Reference samples 1 to 3) with a concentration of 100 mg/mL were prepared by dissolving cyclophosphamide under stirring in the corresponding mixture of liquids. Subsequently the samples were filled into (moisture proof) vials which were subsequently sealed using rubber stoppers. In each case all samples were formulated in triplicates and the assay value was calculated as the average value for said triplicates, respectively.

The stability of the cyclophosphamide in the corresponding samples was measured at a storage temperature of 5° C. after 0 day (T₀), 35 days (T₃₅), 91 days (T₀₉) and 330 days (T₃₃₀). The stability of cyclophosphamide was assessed via determination of assay by high performance liquid chromatography. For the determination of assay the peak area of the active ingredient was compared against an external calibration with a reference substance and is expressed as % amount.

TABLE 1
Assay about stability of cyclophosphamide

| | Assay | Assay | Assay | Assay |
|---|---|---|---|---|
| Formulation | at T₀ | at T₃₅ | at T₉₁ | at T₃₃₀ |
| [V/V] | [%] | [%] | [%] | [%] |
| A: | 90.013 | 94.785 | 95.279 | |
| PG:EtOH | | | | |
| [0.25/0.75] | | | | |
| B: | 90.330 | 94.405 | 95.165 | |
| PS80:PG:EtOH | | | | |
| [0.25/0.25/0.50] | | | | |
| C: | 89.030 | 92.338 | 93.364 | |
| PS80:PG:EtOH | | | | |
| [0.0833/0.333/0.583] | | | | |
| D: | 92.831 | 94.182 | 94.815 | |
| PS80:EtOH | | | | |
| [0.25/0.75] | | | | |
| E | 95.638 | 94.681 | 94.259 | |
| PS80:PG:EtOH | | | | |
| [0.333/0.083/0.583]: | | | | |
| F: | 89.415 | 93.313 | 93.971 | 92.478 |
| PS80:PG:EtOH | | | | |
| [0.083/0.083/0.833] | | | | |
| G: | 92.857 | 95.346 | 94.659 | |
| PS80:PG:EtOH | | | | |
| [0.167/0.167/0.667] | | | | |
| Reference 1: | 88.781 | 93.593 | 94.099 | |
| EtOH | | | | |
| [1.00] | | | | |
| Reference 2: | 87.668 | 90.403 | 94.922 | |
| PS80:EtOH | | | | |
| [0.50/0.50] | | | | |
| Reference 3: | 90.402 | 95.879 | 95.758 | |
| PG:EtOH | | | | |
| [0.50/0.50] | | | | |

EtOH is ethanol, PG is propylene glycol and PS80 is polyoxyethylen-(20)-sorbitanmonooleate (Polysorbat 80).

It was confirmed that in the present mixture of liquids the cyclophosphamide is as stable as in the reference examples for at least 330 days at 5° C.

### Example 2: Accelerated Stability Study

Additional to the above example, a study experiment was performed with different API concentrations in several mixtures of liquids containing different percentage by volume of propylene glycol (PG) and absolute ethanol (EtOH abs.), respectively. The aim of this study was to reach isoconversion of the molecule by stressing the formulation candidates at different temperatures (30° C., 40° C., 50° C., 60° C.) over 30 days. Using the assay data, the ASAPprime software calculates the probability of passing shelf-life specification (90% of assay) at the target storage condition at shelf-life (24 months).

TABLE 1
ASAPprime results

| | Cyclo- | | | Probability of |
|---|---|---|---|---|
| | phosphamide | EtOH | PG | pass at 5° C. |
| Formulation | [mg/mL] | [V/V %] | [V/V %] | [%] |
| 1 | 200 | 80 | 20 | 98.37 |
| 2 | 150 | 90 | 10 | 97.66 |
| 3 | 100 | 80 | 20 | 99.57 |
| 4 | 100 | 100 | 0 | 99.82 |
| 5 | 150 | 90 | 10 | 97.72 |
| 6 | 150 | 90 | 10 | 97.93 |
| 7 | 200 | 100 | 0 | 90.06 |

As can be seen from Table 2 also liquid mixtures containing just component (b1) and (b2) have a high probability to pass the shelf-life specification, i.e. cyclophosphamide is considered to be stable enough even after storage for 2 years (24 months).

1. A parenteral pharmaceutical composition comprising
(a) cyclophosphamide and
(b) a mixture of liquids
wherein the mixture of liquids (b) comprises
(b1) 60 to 99.5 vol % ethanol,
(b2) 0 to 25 vol % of an organic solvent selected from glycerol, ethylene glycol, polyethylene glycol, propylene glycol and mixtures thereof and
(b3) 0 to 20 vol % of liquid surfactant.

2. The pharmaceutical composition according to claim 1, wherein the pharmaceutical composition is a solution.

3. The pharmaceutical composition according to claim 1, wherein the composition is sterile in accordance with European Pharmacopoeia 4.00/2.06.01.00 and/or 4.00/5.01.01.00.

4. The pharmaceutical composition according to claim 1, wherein the pharmaceutical composition has a storage stability at 5° C. of up to 500 days.

5. The pharmaceutical composition according to claim 1, wherein the pharmaceutical composition comprises cyclophosphamide (a) at a concentration of from 50 mg/mL to 350 mg/mL.

6. The pharmaceutical composition according to claim 1, wherein the mixture of liquids (b) comprises
(b1) 70 to 95 vol.-% ethanol,
(b2) 1 to 22 vol.-% of an organic solvent selected from glycerol, ethylene glycol, propylene glycol and mixtures thereof and
(b3) 1 to 15 vol.-% of liquid surfactant.

7. The pharmaceutical composition according to claim 1, wherein the organic solvent (b2) is propylene glycol.

8. The pharmaceutical composition according to claim 1, wherein the liquid surfactant (b3) is polyoxyethylene (20)-sorbitan monooleate.

9. The pharmaceutical composition according to claim 1, wherein the pharmaceutical composition is a concentrate for infusion.

10. The pharmaceutical a composition according to claim 1 being a veterinarian composition.

11. An injection syringe, a cartridge, a container or a vial containing the pharmaceutical composition according to claim 1 for single or multiple dosing.

12. A kit of parts comprising the pharmaceutical composition according to claim 1 in a vial, cartridge or container and an injection device.

13. The pharmaceutical composition according to claim 1 for use in the treatment of cancer as well as nephrotic syndrome, granulomatosis with polyangiitis and following organ transplant.

14. The pharmaceutical composition for use according to claim 13, wherein the cancer is selected from lymphoma, multiple myeloma, leukemia, ovarian cancer, breast cancer, small cell lung cancer and sarcoma.

15. A method for preparing the parenteral pharmaceutical composition according to claim 1, comprising the steps of
(i) mixing liquids (b1), (b2) and (b3), and
(ii) adding cyclophosphamide (a) to the mixture from step (i) to obtain the parenteral pharmaceutical composition.
